SEQUENCE LISTING (2 sequences)

SEQ ID NO: 1 - protein, 293 aa, Homo sapiens [sequence omitted]

SEQ ID NO: 2 - protein, 159 aa, Homo sapiens
GHVGLGVKCSKEVATAIRGAIILAKLSIVPVRRGYWGNKIGKPHTVPCKVTGRCGSVLVRLIPAPRGTGIVSAPVPKKLLMMAGIDDCYTSARGCTATLGNFAKATFDAISKTYSYLTPDLWKETVFTKSPYQEFTDHLVKTHTRVSVQRTQAPAVATT

(19) United States
(12) Patent Application Publication
(10) Pub. No.: US 2026/0308529 A1
(43) Pub. Date: Oct. 8, 2026

(54) COMPOSITIONS AND METHODS FOR PROPOGATING INSULIN AND GLUCAGON SECRETING CELLS FROM TYPE 1 DIABETIC PANCREATIC TISSUE AND THERAPEUTIC USES THEREOF

(71) Applicant: IMAGINE PHARMA LLC, PITTSBURGH, PA (US)

(72) Inventors: NGOC THAI, PITTSBURGH, PA (US); JONATHAN POLLETT, PITTSBURGH, PA (US); RITA BOTTINO, PITTSBURGH, PA (US)

(21) Appl. No.: 19/171,936

(22) Filed: Apr. 7, 2025

(51) Int. Cl. C12N 5/071; A61K 35/39; A61K 38/26; A61K 38/28

(52) U.S. Cl. CPC C12N 5/0677; A61K 35/39; A61K 38/26; A61K 38/28; C12N 2501/335; C12N 2501/599; C12N 2510/02

(57) ABSTRACT

Disclosed herein are compositions and methods for generating compositions comprising cell-based therapeutics useful for treating pancreatic disorders, including Type 1 diabetes.

### CROSS REFERENCE TO RELATED APPLICATION

This application claims priority to U.S. patent application Ser. No. 18/294,669 filed on Feb. 20, 2024, and U.S. provisional patent application No(s). 63/247,252, filed on Sep. 22, 2021 and 63/337,137, filed on May 1, 2022, the contents of which are herein incorporated by reference in their entirety.

### SEQUENCE LISTING

This application includes a Sequence Listing, which contains amino acid sequences that are integral to the invention described herein. The Sequence Listing is provided in an electronic format (e.g., .xml file) and is incorporated by reference into the specification in its entirety. The sequences are identified by specific sequence identifiers (SEQ ID NOs), as detailed below in Table 1. The Sequence Listing is submitted in compliance with the requirements set forth in 37 C.F.R. § 1.821 and is an essential part of the disclosure.

### BACKGROUND

Cell-based therapies offer the promise of treating and altering the course of pancreatic disorders, such as Type 1 diabetes (T1D), which cannot be addressed adequately by existing therapies, yet cell-based therapies present myriad issues, mainly related to safety and efficacy and scalability of manufacture. Many of the problems associated with cell-based therapies are described in Engineering the next generation of cell-based therapeutics by Bashor, C. J., et al., Nat Rev Drug Discov (2022) (and available online at https://doi.org/10.1038/s41573-022-00476-6).

### SUMMARY OF THE DISCLOSURE

Disclosed herein are compositions and methods for generating compositions comprising cell-based therapeutics useful for treating pancreatic disorders, including Type 1 diabetes. In one embodiment, a composition as disclosed herein comprises an insulin and glucagon secreting cell population generated from non-insulin secreting pancreatic cells collected via needle biopsy from a Type 1 diabetic donor pancreas. In another embodiment, a composition as disclosed herein comprises an insulin- and glucagon secreting cell population generated from pancreatic cells collected via needle biopsy from a patient or donor suffering from chronic pancreatitis. In one embodiment, non-insulin secreting Type 1 diabetic pancreatic cells are treated, in vitro, with an islet cell culture medium comprising a base medium and an effective amount of a polypeptide according to an amino acid sequence listed in SEQ ID 1 or 2, wherein treatment causes the treated cells to differentiate and propagate into a population of islet progenitor cells that secrete both insulin and glucagon in response to stimuli and are CD133 positive. The resulting insulin and glucagon secreting progenitor cells can be propagated to a desirable cell count for subsequent use in transplantation or injection and as a cell-based therapeutic for Type 1 diabetes or chronic pancreatitis. The cellular composition comprising an effective amount of an insulin and glucagon secreting progenitor cell population may be administered to a subject by infusion, injection, transplantation, intra portal delivery, or by other suitable delivery means such as with a medical device, as method for restoring secretion of insulin and glucagon in response to stimuli.

The compositions and methods disclosed herein have implications for producing large volumes of insulin and glucagon secreting pancreatic cells useful for cell-based therapies and cellular transplantations, namely autologous or allogenic transplantation for the treatment of Type 1 diabetes or chronic pancreatitis.

Also disclosed herein is a method of treating a pancreatic disorder, such as Type 1 diabetes or pancreatitis, comprising administering to a subject in need thereof, a therapeutically effective amount of a composition comprising an insulin and glucagon secreting pancreatic cell population, wherein the insulin and glucagon secreting pancreatic cell population is generated by treating pancreatic cells collected from diseased pancreatic tissue (for example, from a Type 1 diabetic subject or one suffering from chronic pancreatitis), such as via needle biopsy, with an islet cell culture media comprising a base medium and a peptide comprising an amino acid sequence according to SEQ ID 1 or 2. The composition, when administered to a subject in need thereof, provides delivery of healthy pancreatic progenitor cells to a target site in the subject, wherein the healthy pancreatic progenitor cells are capable of producing insulin and glucagon in response to stimulation.

The composition comprising a therapeutically effective amount of insulin and glucagon secreting progenitor cells generated by the methods disclosed herein may be used as an autologous or allogenic cell based therapeutic to supplement the loss of insulin production or replace insulin production in patients with Type 1 diabetes, or with other diseases characterized by severe insulin deficiency, such as after total or partial pancreatectomy, with and without autologous or allogenic islet transplantation.

In one embodiment, compositions may be prepared for transplantation by supplementing the compositions with human serum albumin and/or human serum from the recipient prior to administration.

In another embodiment, an islet cell culture medium useful for stimulating growth, propagation and differentiation of insulin and glucagon secreting cells from pancreatic cells derived from Type 1 diabetic pancreatic tissue comprises a base medium and an effective amount of a polypeptide, wherein the polypeptide comprising an amino acid sequence according to one or more of SEQ ID NO. 1-2 (listed in Table 1), or active fragment thereof. In one embodiment, the polypeptide comprises an amino acid sequence having at least 50% sequence identity to the amino acid sequence set forth in SEQ ID NO. 01; in another embodiment, the polypeptide has at least 50%, 55%, 60%, 65%, 70%, 75%, 80%, 85%, 90%, 95%, 98% or 99% sequence identity to the amino acid sequence set forth in SEQ ID NO. 1. Alternatively, the polypeptide comprises an amino acid sequence having at least 50% sequence identity to the amino acid sequence set forth in SEQ ID NO. 2; in another embodiment, the polypeptide has at least 50%, 55%, 60%, 65%, 70%, 75%, 80%, 85%, 90%, 95%, 98% or 99% sequence identity to the amino acid sequence set forth in SEQ ID NO. 2.

In yet another embodiment, a cellular composition comprises a population of insulin and glucagon secreting cells generated by treatment of isolated Type 1 diabetic pancreas tissue with an islet cell culture medium comprising a base medium and an effective amount of a polypeptide according to SEQ ID NO. 1 or 2 or active fragment thereof; further comprising measuring the response of the cells to glucose; wherein the cellular composition comprises a population of cells capable of secreting insulin and glucagon in response to appropriate stimuli.

In another embodiment, a method of manufacturing a cellular composition comprises applying, in vitro, an islet cell culture medium comprising a base medium and an effective amount of a polypeptide according to SEQ ID NO. 1 or 2, or active fragment thereof, to human pancreatic tissue collected from a Type 1 diabetic; incubating the cells in the islet cell culture medium; screening the incubated cells for one or more cell markers selective for CD133 and insulin; and collecting the cells identified by screening as CD133 and insulin-positive from the cultured cell population; continuing to grow the cultured cells until a desired quantity of cells are propagated.

In yet another embodiment, cellular compositions comprising insulin and glucagon secreting cells derived from T1D pancreatic tissue are packaged or encapsulated for administration or implantation into a mammal for in vivo therapy, specifically to restore insulin production and secretion. The cellular compositions may be packaged as a delivery solution, or in a delivery vehicle, and administered by implantation, injection or infusion, whether administration is systemic, localized or directed to a target site.

In yet another embodiment, a method of treating a pancreatic disorder, wherein the pancreatic disorder is characterized by an insufficient production of insulin, in a mammal, comprises: culturing, in vitro, a population of insulin and glucagon secreting cells from pancreatic tissue collected from a Type 1 diabetic donor pancreas, in an islet cell culture medium comprising a base medium and an effective amount of a polypeptide according to SEQ ID NO. 1 or 2 or active fragment thereof, whereby a population of CD133 positive, insulin and glucagon secreting cells are produced; further comprising isolating and expanding the population to generate a predominantly (at least 60% or greater) insulin and glucagon secreting cell population; and further comprising collecting the insulin and glucagon secreting cells and suspending the collected cells in a physiologic buffer, such as phosphate buffered saline (PBS) or Hanks Balanced Salt Solution (HBSS), and implanting or injecting into a mammal a cellular composition comprising the insulin and glucagon secreting cells in suspension with physiologic buffer. In one embodiment, the composition may be delivered as an aqueous solution, a suspension, an encapsulation, a microencapsulation, and/or an encapsulated, or semi-solid formulation; wherein the composition may be delivered to the mammal via one or more of an injection, infusion, omental or peritoneal pouch, surgical implantation, or via packaging the composition as part of a device to a target site in the mammal.

In another embodiment, a cellular composition comprises an insulin and glucagon secreting cell population further comprising one or more of a buffer, a pharmaceutically acceptable carrier, a pharmaceutically acceptable additive, an antibiotic or other pharmaceutical agent.

### DETAILED DESCRIPTION OF THE DRAWINGS

The compositions and methods disclosed herein are further described by the accompanying figures. The term “IPCs” are used in the figures to refer to the insulin-producing cells (IPCs) described and claimed herein.

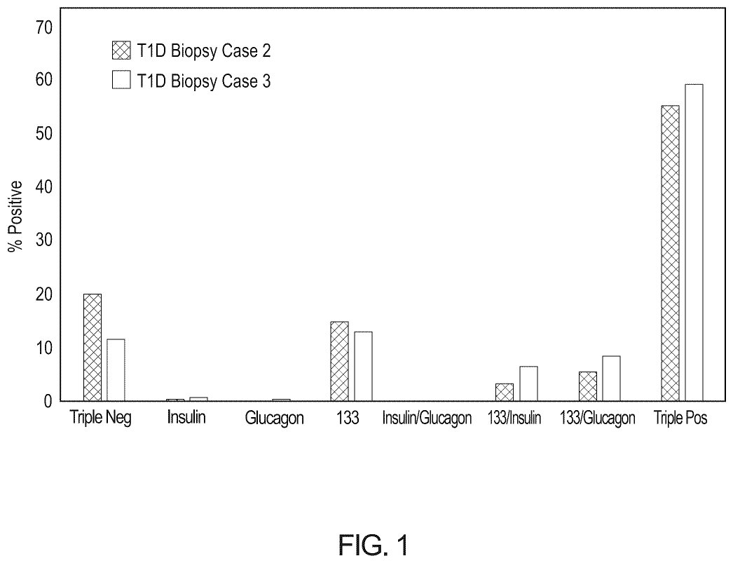

FIG. 1 shows that T1D-derived insulin and glucagon secreting cells propagated according to methods herein are greater than 50% triple positive for CD133, insulin and glucagon.

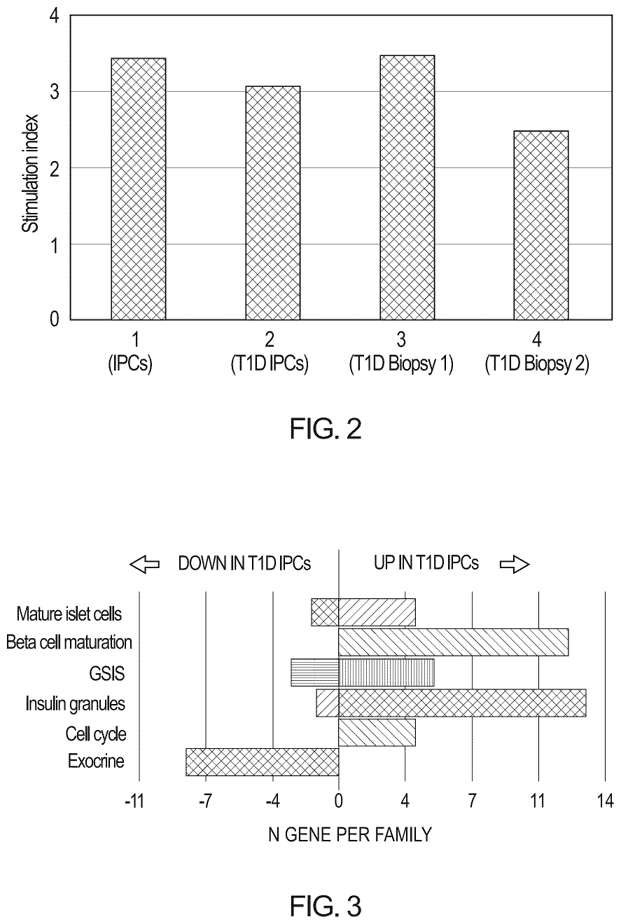

FIG. 2 shows T1D pancreatic tissue cultured with islet cell culture medium comprising a peptide according to SEQ ID NO. 1 or 2 produce cells that secrete insulin in response to glucose stimulation, as shown by the stimulation index, which is the ratio between insulin secretion under high glucose conditions vs basal release under unstimulated conditions. A value above 2 represents glucose responsiveness in the cells. Sample 1 is a cell population propagated from normal pancreatic tissue according to methods herein; Samples 2-4 are cell populations propagated from T1D pancreatic tissue according to methods herein.

FIG. 3 shows the down regulation and up regulation of gene (families) associated with pancreatic function in a single T1D biopsy-derived cell preparation compared to native pancreatic tissue. Each family of genes includes 5 to 13 genes. As the figure shows, cell populations comprising insulin and glucagon secreting cells generated according to the methods herein exhibit upregulation of gene families essential for mature islet cells, beta cell maturation, GSIS, insulin granules and cell cycle.

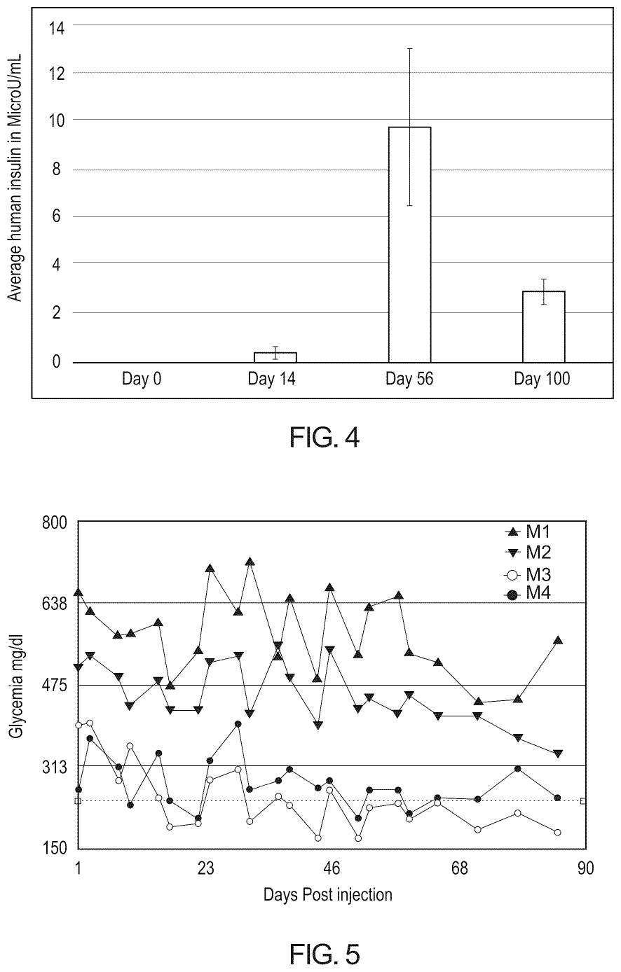

FIG. 4 shows serum insulin levels following transplantation in streptozotocin (STZ, a β-cell-specific toxin that induces irreversible damage to pancreatic islets and induces diabetes) treated mice of cellular compositions comprising insulin and glucagon propagated according to methods herein. The cellular compositions were shown to promote the secretion of human insulin in vivo, which was present in the serum for up to 100 days of STZ mice treated with cellular compositions disclosed herein.

FIG. 5 shows that insulin and glucagon secreting cells generated from Type 1 diabetic (T1D) cells propagated according to methods herein can normalize blood glucose levels upon injection in a STZ diabetic mouse model. M1-4 refer to STZ mouse sample numbers, ie, Mouse-1, Mouse-2, Mouse-3, Mouse-4.

### DETAILED DESCRIPTION OF THE DISCLOSURE

The following terms are used in this disclosure to describe different embodiments. These terms are used for explanation purposes only and are not intended to limit the scope for any aspect of the subject matter claimed herein.

As used herein “SEQ ID NO 1 or 2” refers to a protein, polypeptide, peptide fragment, or analogue thereof, and including any modification thereto, having an amino acid sequence having at least 85%, 90%, 95%, 98% or 99% sequence identity to the amino acid sequence according to SEQ ID NO 1 or 2 (See Table 1). Also contemplated is a peptide fragment, or analogue thereof, and including any modification thereto, having an amino acid sequence having at least 85%, 90%, 95%, 98% or 99% sequence identity to the amino acid sequence according to SEQ ID NO(s): 1 and 2. It has been demonstrated by in vitro studies with polypeptides according to SEQ ID NO 1 and SEQ ID NO 2 that treatment of cells (keratinocytes, enterocytes, islet, endothelial and pneumocyte cells) with a polypeptide according to SEQ ID NO 1 or SEQ ID NO 2 added to cell culture medium causes stimulation and increase in cell growth, resulting in viable progenitor cells, as measured by the percent of CD133 positive cells in culture and MTT cell proliferation assays (Sigma Aldrich Cell Proliferation Kit). These progenitor cells can be regenerated and propagated into the billions.

As used herein, the term “insulin and glucagon secreting cells” or “insulin and glucagon secreting islet cells” or “insulin and glucagon secreting progenitor cells” are used interchangeably herein to refer to the cellular composition comprising insulin and glucagon secreting cells and/or cell population(s), which are generated from non-insulin secreting T1D pancreatic cells according to the methods described herein, and which are positive for the cell markers: CD133, insulin, glucagon, and that produce insulin and glucagon in response to stimuli, and are further characterized by cell markers PDX-1, SST, IIAP, Pax4, Pax6, Nkx2, Nkx6, NeuroD1, MafA, MafB.

The term “propagation” refers to an increase in the number of cells present in a culture as a result of cell division.

As used herein “culture”, “cultured” or “culturing” refers to removal or isolation of cells from an environment (such as in a host mammal) and their subsequent growth in a favorable artificial environment in vitro. “Cultured cells” is intended to include sub-cultured (i.e., passaged) by transferring the cells to a new vessel with fresh growth medium to provide more room for continued growth, differentiation and/or propagation. Reference to “pancreatic cells” include those cells normally found in the pancreas of a mammal, and include pancreatic islet cells, e.g., glucagon-synthesizing alpha cells, insulin-producing beta cells, and any combination thereof.

The term “target site” as used herein refers to a region in the recipient host (a mammal, preferably human) that requires treatment or supplementation. The target site can be a single region within a specific organ or can be multiple regions in the host. In some embodiments, the supplementation or replacement results in the same physiological response as normal tissue, such as pancreatic tissue, whether or not targeting the pancreas.

As used herein, the terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

As used herein, an “effective amount” refers to an amount that is sufficient to achieve the stated effect. A therapeutically effective amount to treat a condition is an amount capable of achieving a clinically relevant end-point in a patient or patient population. As non-limiting examples, administration of an effective amount of a composition comprising insulin and glucagon secreting cells is an amount of approximately 1.2 to about 2.5×10⁶ cells/kg, or greater than 200×10⁶ cells, in order to produce sufficient insulin to cause a reduction in blood glucose levels to approximately 100 to 125 mg/dl (5.6 to 6.9 mmol/L), or to under 250 mg/dl. Other ranges include approximately 3×10⁶ cells to about 25×10⁶ cells per kg of body mass; or approximately 5×10⁶ to about 10×10⁶ millions cells/kilo. The appropriate dose of the composition may depend on the route of administration, such as injection or infusion or transplantation, and may depend on the subject being treated as well as the severity of the condition to be treated. Using scaling methods, such as allometric scaling, it is possible to predict suitable and exemplary dosage ranges for the administration of compositions, as disclosed herein, to adult humans. Dose scaling is an empirical approach, is well characterized and understood in the art. This approach assumes that there are some unique characteristics on anatomical, physiological, and biochemical process among species, and the possible difference in pharmacokinetics/physiological time is, as such, accounted for by scaling. As one example, not intending to be limiting, the human pancreas, based on the literature, has between 6×10⁵ to about 2×10⁶ islets; hence there are approximately 600×10⁶ islet cells in the normal human pancreas, with half of them being beta cells. Considering that an islet mass of 30% is sufficient to maintain normoglycemia, a dose of 1.2×10⁶ of the insulin and glucagon secreting cells as disclosed herein would be expected to sufficiently replace the insulin producing capacity of a non-diseased human pancreas.

As used herein, the term “sequence identity” refers to the identity between two or more amino acid sequences expressed in terms of the identity or similarity between the sequences. Sequence identity can be measured in terms of percentage identity; the higher the percentage, the more identical the sequences are. The percentage identity is calculated over the entire length of the sequence. Homologs or orthologs of amino acid sequences possess a relatively high degree of sequence identity when aligned using standard methods. This homology is more significant when the orthologous proteins are derived from species which are more closely related (e.g., human and mouse sequences), compared to species more distantly related (e.g., human and C. elegans sequences). Methods of alignment of sequences for comparison are well known in the art. Various programs and alignment algorithms are described in: Smith & Waterman; Needleman & Wunsch, J. Mol. Biol. 48:443, 1970; Pearson & Lipman, Proc. Nat. Acad Sci. USA 85:2444, 1988; Higgins & Sharp, Gene, 73:23744, 1988; Higgins & Sharp, CABIOS 5:151-3, 1989; Carpet et al., Nuc. Acids Res. 16:10881-90, 1988; Huang et al. Computer Appls. in the Biosciences 8, 155-65, 1992; and Pearson et al., Meth Mol. Bio. 24:307-31, 1994. Altschul et al., J. Mol. Biol. 215:403-10, 1990, presents a detailed consideration of sequence alignment methods and homology calculations. The level of sequence identity may be determined using the NCBI Basic Local Alignment Search Tool (BLAST) (Altschul et al., J. Mol. Biol. 215:403-10, 1990), which is available from several sources, including the National Center for Biological Information (NCBI, National Library of Medicine, Building 38A, Room 8N805, Bethesda, Md. 20894, US) and on the Internet.

It will be understood that a numerical value may be associated with a certain amount of experimental error. Thus, recitation of the qualifier “about” (or “approximately”) prior to a numerical error is meant to embody the experimental error that may be associated with the recited numerical value. To the extent that a numerical value obtained experimentally is not preceded by the expression “about” (or “approximately”) does not mean that the numerical value is not associated with a certain amount of experimental error.

Representative cultures of the insulin and glucagon secreting cells characterized herein have been deposited with ATCC on Sep. 7, 2022 [Accession Number______] under the terms of the Budapest Treaty. Cultured cells, propagated cells, isolated cells, and the like may be protected from external mutagenic stimuli, such as, for example UV radiation.

Utilizing the methods disclosed herein, it has been determined that 30 days post isolation of T1D pancreatic tissue, a single pancreas can product 77 billion insulin and glucagon secreting (islet) cells; 2 trillion cells by day 60, which is enough cells to infuse 100-150 patients in need of treatment (depending on the severity of illness or dosage used), or bank (freeze/store) cells for future expansion and re-infusion.

TABLE I
SEQ ID NO(s)

| | |
|---|---|
| SEQ ID NO. 01 | MADDAGAAGGPGGPGGPGMGNRGGFRGGFGSGIRGRGRGRGRGRGRGRGARGG |
| (293 aa) | KAEDKEWMPVTKLGRLVKDMKIKSLEEIYLFSLPIKESEIIDFFLGASLKDEVLKIMPVQK |
| | QTRAGQRTRFKAFVAIGDYNGHVGLGVKCSKEVATAI RGAIILAKLSIVPVRRGYWGNK |
| | IGKPHTVPCKVTGRCGSVLVRLIPAPRGTGIVSAPVPKKLLM MAGIDDCYTSARGCTAT |
| | LGNFAKATFDAISKTYSYLTPDLWKETVFTKSPYQEFTDHLVKTHTRVSVQRTQAPAVATT |
| SEQ ID NO. 2: | GHVGLGVKCSKEVATAIRGAIILAKLSIVPVRRGYWGNKIGKPHTVPCKVTGRCGSVLVR |
| (159 aa) | LIPAPRGTGIVSAPVPKKLLMMAGIDDCYTSARGCTATLGNFAKATFDAISKTYSYLTPD |
| | LWKETVFTKSPYQEFTDHLVKTHTRVSVQRTQAPAVATT |

Amino acid residues of the active agents may be post-translationally modified or conjugated with other functional or non-functional molecular groups. See, e.g., Guo et al. Mol. Biosyst. 7 (7): 2286-2295, 2011, describing generally antagonistic citrullination and methylation of human ribosomal protein S2 (e.g., SEQ ID NO. 1). Naturally, such modified amino acid residues are included in the amino acid sequences and within the scope of the active agents described herein.

The polypeptides and/or polypeptide fragments according to, for example, SEQ ID NO(s): 1 and 2 may be produced under conditions known in the art for protein production, such as production in bacteria, yeast or by synthetic means, or as described in U.S. patent application Ser. No. 15/811,060.

In one embodiment, the cellular compositions may be packaged as a delivery solution, or in a delivery vehicle comprising a medical device, and may be administered by implantation, injection, or infusion, whether administration is parenteral, systemic, localized, or directed to a target site. In one embodiment, encapsulation of in vitro-generated insulin and glucagon secreting islet cells and implantation into a mammal have been previously characterized in the art (see, for example, Altman, et al., 1984, Trans. Am. Soc. Art. Organs 30:382-386, and U.S. Pat. No. 6,703,017 B1, herein incorporated by reference)—and would be suitable for the insulin and glucagon secreting cells generated according to the methods disclosed herein. Preferably, the encapsulant is hypoallergenic, is easily and stably situated in a target tissue, and provides added protection to the implanted cellular composition, to protect and prevent from the destruction of the implanted cells.

The appropriate implantation dosage in humans can be determined from existing information relating to ex vivo islet transplantation in humans, further in vitro and animal experiments, and from human clinical trials. From data relating to transplantation of ex vivo islets in humans, it is expected that about 8,000-12,000 islets per patient kg may be required. Assuming long-term survival of the implants following transplantation, less than the number of naturally occurring islets (about 2 million in a normal human adult pancreas), or possibly even less than the amount used in ex vivo islet transplantation may be necessary.

In one embodiment, the cellular compositions are of therapeutic benefit for treating a pancreatic disorder, wherein the pancreatic disorder is hyperglycemia, Type 1 diabetes, or chronic pancreatitis, in a mammal, which comprises: administering a therapeutically effective amount of the insulin and glucagon secreting cell population, thereby providing a treatment for the pancreatic disorder.

In one embodiment, the composition comprising a therapeutically effective amount of an insulin and glucagon secreting cell population may be formulated as an aqueous solution, a suspension, an encapsulation, a microencapsulation, and/or an encapsulated, or semi-solid formulation; wherein the composition may be delivered to the patient in need via one or more of an injection, infusion, omental or peritoneal pouch, port, surgical implantation, or via packaging the composition as part of a device to a target site in the mammal.

In one embodiment, a composition comprises an insulin and glucagon secreting progenitor cell population further comprising one or more of a pharmaceutically acceptable excipients, and/or one or more of a pharmaceutically acceptable additive and/or one or more a pharmaceutical agent. Suitable excipients and additives include, but are not limited to buffering agents such as PBS, or HBSS, amino acids, stabilizer or bulking agents, surfactants, antimicrobial/preservatives, antifungal agents, metal ions/chelators, polymers, polyanions, salts, sugars, cyclodextrin based excipients, lyoprotectants, solubilizing agents, antioxidants, complexing agents, anti-adhesive agents, dispersing agents, serum additives.

In another embodiment, the disclosure provides a method for treating a mammal, preferably a human, suffering from, or at risk of developing Type 1 diabetes or severe pancreatitis, which comprises: removing pancreatic tissue from the mammal; culturing the excised pancreatic tissue in vitro to propagate a population of insulin and glucagon secreting islet cells; and implanting, transplanting, infusing, injecting, or otherwise inserting the population of insulin and glucagon secreting islet cells, alone or together with a medical or delivery device, into the mammal.

### EXAMPLES

The following Examples are offered by way of illustration and not by way of limitation with respect to subject matter claimed herein.

Cell cultures performed under the Examples were incubated at 37° C. under standard CO (5%) conditions; culturing (plating, splitting of cells) was performed using standard aseptic techniques and conditions in a vertical laminar flow hood. Unless stated otherwise, cells (including controls) were cultured in the islet cell culture media described in Table 2 and 3. Cells were split when they reached confluency of approximately 70-80% in culture.

In one embodiment, the splitting technique involved removal of supernatant from the culture plates (supernatant was preserved). Plates were then washed with 2-5 ml PBS (wash was preserved). Cells were detached using approximately 3-5 ml of trypsin (such as available from Sigma-Aldrich) by incubating the cells in the presence of trypsin at 37° C. for approximately 3-5 minutes until cells detached. Plates were then washed a second time with PBS. The trypsinized cells, as well as the preserved PBS washes and collected cell culture supernatant, were then centrifuged at 300 g for 7 minutes at 4° C.

In one embodiment, the resulting supernatant was decanted, and the pellet resuspended in 2 ml PBS, and recentrifuged. The supernatant was then removed, and the pellet resuspended in culture medium containing SEQ ID 1 or 2 and re-plated at a cell density of ~1000 cells/cm². While the Examples may refer to cell culture plates, it will be understood that cell culture flasks are an acceptable alternative to plates.

TABLE 2
Islet Cell Culture Medium

| MEDIUM COMPONENT | AMOUNT | |
|---|---|---|
| CMRL medium (or other | 500 | mL |
| similar equivalent nutrient | | |
| media which could be | | |
| prepared and utilized by | | |
| those skilled in the art) | | |
| L-glutamine | 2 | mmol |
| Ciprofloxacin | 2 | mg/L |
| Amphotericin B | 0.1 | mg/L |
| Penicillin | 100,000 | units/L |
| Streptomycin | 100,000 | micrograms/L |
| SEQ ID 1 or 2 | 3-20 | μg/mL |

| | |
|---|---|
| Fetal calf serum (FCS) (or | 10% |
| horse, or bovine serum) | |
| Human Serum | 10% |

TABLE 3
CONTROL Medium

| MEDIUM COMPONENT | AMOUNT | |
|---|---|---|
| CMRL medium (or other | 500 | mL |
| similar equivalent | | |
| nutrient media which | | |
| could be prepared and | | |
| utilized by those skilled | | |
| in the art) | | |
| Ciprofloxacin | 2 | mg/L |
| Amphotericin B | 0.1 | mg/L |
| Penicillin | 100,000 | units/L |
| Streptomycin | 100,000 | micrograms/L |

| | |
|---|---|
| Fetal calf serum (FCS) | 10% |
| (or horse or bovine serum) | |
| Human Serum | 10% |

### Example 1

Method of generating an insulin and glucagon secreting pancreatic cell population from non-insulin-producing pancreatic tissue collected by needle biopsy from a Type 1 Diabetic donor.

Human pancreatic tissue was collected via needle biopsy from a Type 1 diabetic (T1D) donor patient (58-year-old female; 53 years diabetic). Biopsies of 1×1 mm3 were obtained from the donor pancreas (procured from a center for organ recovery and education) preserved on ice in a commercially available solution (sold under names such as Viaspan, Belzer UW, Bel-Gen or StoreProtec).

The collected T1D tissue was then cultured in an islet cell culture medium (See Table 2) comprising CMRL (such as Mediatech #99-663-CV Transplant Medium (CMRL 1066) without phenol red) supplemented with L-glutamine (2 mmol), Ciprofloxacin (2 mg/L), Amphotericin B (0.1 mg/L) Penicillin (100,000 units/L) and Streptomycin (100,000 micrograms/L), and a polypeptide according to SEQ ID NO. 1 or 2 (in a range of 3-20 μg/ml, and specifically 10 μg/ml), with fetal calf serum (FCS) (10%) and human serum (10%). Control culture medium is one which comprises CMRL supplemented with L-glutamine (2 mmol), Ciprofloxacin (2 mg/L), Amphotericin B (0.1 mg/L) Penicillin (100,000 units/L) and Streptomycin (100,000 micrograms/L) and fetal calf serum (FCS) (10%) and human serum (10%) (without the addition of a polypeptide according to SEQ ID NO. 1 or 2). Standard tissue/cell culture conditions (37° C., 5% CO₂) were used.

Tissue is cultured on plates or in flasks coated with an attachment factor mixture (AFM) comprising Collagen Type I (Collagen from rat tail, Sigma-Aldrich C3867) and Endothelial Cell Attachment Factor (ECAF, Sigma-Aldrich E9765). Various ratios of ECAF and collagen may be used, including but not limited to a 50/50 ratio of collagen to ECAF. Briefly, plates (or flasks) were prepared by applying a thin layer of AFM (between 3-10 ml) to the plates, and after setting for 30 minutes the excess AFM was removed. The plates were allowed to dry for 45 minutes in a hood. Prior to use the plates were washed with PBS to remove any potential contaminants. The collected tissue was incubated on the AFM-treated plates in islet cell culture medium supplemented with a polypeptide according to SEQ ID NO. 1 or 2 until cells began to mobilize and proliferate (approximately 10 to 20 days).

After 12-15 days in culture the biopsy-derived cells were mobilized and began to adhere to the dish and to proliferate. During a subsequent 4-6-week period, the adherent cells continued to proliferate, became confluent and continue to multiply, doubling every 3 days. The biopsy-derived cells in culture exhibited morphological similarities to cellular compositions comprising insulin secreting cells generated from non-diabetic donors, previously characterized in US 2021/0205371, published Jul. 8, 2021. Similarly, the cells formed islet-like cell clusters with size consistent with that of islets of Langerhans and were shown to secrete insulin in response to stimulation with glucose.

The resulting cell cultures derived from T1D pancreatic biopsy tissue were also assayed for the expression of CD133, as well as intracellular insulin and glucagon expression, by fluorescence activated cell sorting (FACS) using a flow cytometry instrument (Becton Dickinson FACS Aria cell sorter). The cultured cells were initially labeled for CD133 expression and then fixed and permeabilized with FOXP3 Fixation/Permeabilization Buffer, as per the manufacturer's instructions, and stained with conjugated fluorometric antibodies for glucagon and intra-cellular insulin, respectively. FACS analysis followed FOXP3 Fixation Permeabilization and staining with conjugated fluorometric antibodies. T1D pancreas biopsy-derived cells cultured in islet cell culture medium comprising a peptide according to SEQ ID NO. 1 or 2 were found to be positive for CD133, glucagon and insulin (referred to herein as “triple positive”), specifically, 48-73% triple positive for insulin, glucagon and CD133, 26-42% double positive for glucagon and CD133, 14-18% triple negative for insulin, glucagon and CD133, 9-23% single positive for glucagon, and 0-7% single positive CD133, and negative for insulin and glucagon; negative for insulin and CD133; and negative for insulin. It was determined that, overall, the cultured cell population was greater than 65% positive for insulin, CD133 and glucagon (triple positive). (See FIG. 1) Example 2

An insulin and glucagon secreting pancreatic cell population generated from non-insulin producing pancreatic tissue collected by needle biopsy from a Type 1 Diabetic donor secretes insulin in response to glucose stimulation in vitro.

To test glucose responsiveness of the insulin and glucagon secreting cells propagated from T1D biopsy tissue (See Example 1), the insulin and glucagon secreting cells were subjected to a glucose stimulation insulin secretion assay. Approximately 1×10⁶ cells/well were plated in a 6-well dish and underwent 2 stimulus conditions to assess insulin secretion. The cells were incubated for 30 minutes with either: (1) islet cell culture medium (See Table 2); or (2) islet cell culture medium supplemented with a higher glucose concentration (final concentration 16.7 mM, as a stimulus for insulin secretion). Following incubation, the supernatant was stored at −20° C. until undergoing a standard ELISA assay for insulin quantification. The cells cultured in the islet cell culture medium supplemented with a higher glucose concentration were shown to secrete higher insulin amounts than those cells treated with standard islet cell culture medium (unstimulated controls). There was a relative increase in insulin secretion following glucose stimulation compared to the unstimulated controls, more specifically, higher insulin amounts (95+/−11 pMol/L) were seen in the stimulated cultures compared to those cells treated with standard islet cell culture medium (32+/−7 pMol/L the “unstimulated control”). See FIG. 2, which shows the stimulation index (the measure of the ratio between insulin secretion with high glucose versus basal release) of the cells treated with islet cell culture medium supplemented with high glucose, compared to unstimulated controls.

### Example 3

Characterization and mRNA analysis of insulin and glucagon secreting cells derived from T1D donor tissue.

Using RNA sequencing methods, characteristics of several cell populations were determined, including pancreatic tissue from a deceased donor, insulin and glucagon secreting cells generated by methods described herein, and denovo-pseudo-islets from the same deceased donor. Characteristics of the various cell types were assessed using an Illumina® NovaSeq™ platform. Markers assessed were: insulin, glucagon, PDX-1, SST, IIAP, Pax4, Pax-6, NKx2, Nkx6, NeuroD1, MafA, and MafB. It was also determined that cellular compositions comprising insulin and glucagon secreting cells generated from T1D-derived pancreatic tissue exhibited a substantial decrease in markers of exocrine function (AMY and CTRC), which are expressed in the native pancreas. Concurrently, markers of proliferation PCNA and CCND1 (cyclin family) increased in expression in the cellular compositions, potentially signifying de-differentiation to motile, proliferating cells, consistent with the observation of cell expansion in vitro. Following longer periods of culture (~20 days or more), the insulin and glucagon secreting cells generated from pancreatic tissue collected by needle biopsy from a T1D donor underwent morphological re-arrangement and spontaneously generated de novo-pseudo-islets. These islet-like structures were characterized by a significant increase in expression of an endocrine progenitor and islet signature markers that included insulin, glucagon, PDX-1, SST, IIAP, Pax4, Pax-6, NKx2, Nkx6, NeuroD1, MafA, and MafB, while exhibiting downregulation of the cell proliferation pathways. Moreover, IGFBP1 a marker of β-cell regeneration, was found expressed at higher levels in pseudo-islets when compared to the cellular compositions comprising insulin and glucagon secreting cells, whereas the stem cell markers LY6E and PROM1 were more highly expressed in the cellular compositions, suggesting the maturation to a more differentiated endocrine progenitor cell population committed to generate alpha, beta and delta cells.

Microarray mRNA profile of the cells confirmed a gene profile compatible with a pancreatic endocrine islet cell population, with expression of insulin, and glucagon. Furthermore, these T1D-derived cell populations also expressed the pancreatic transcription factors PDX1, Nkx6, ngn3, NeuroD, and MafA and MafB, as well as the islet neogenesis factor nestin, the glucose transporter Glut-2, the secretory product of β-cells IAPP, and somatostatin which, is secreted by islet δ-cells. These findings suggest that the T1D-derived cell populations carry all factors necessary for islet neogenesis. See FIG. 3, which shows an overview of the upregulation and downregulation of gene families in the insulin and glucagon secreting cells derived from T1D pancreatic tissue.

### Example 4

Method of increasing insulin secretion in vivo via transplantation into a host animal of cellular compositions comprising insulin and glucagon secreting cells.

To test the effectiveness of the T1D-derived insulin and glucagon secreting cells as both a therapeutic and a method for transplantation, four STZ-treated mice (NOD-SCID, 5-6 weeks old; Jackson Laboratory, Bar Harbor, ME) were injected with approximately 2.5×10⁶ T1D-derived biopsy-derived insulin and glucagon secreting cells (cells were counted using a Neubauer Chamber) twice, one week apart (total of 2 doses). Follow up was 30 days. Blood samples were obtained via tail vein after 14 days post first dose, and at the end of the follow up. Serum was obtained and stored at −20° C. until used to measure human insulin and human C-peptide concentrations by ELISA (Abcam and Alpco, respectively).

T1D-derived Insulin and glucagon secreting cells were generated using methods described herein, for example by culturing T1D pancreatic tissue in a culture medium comprising a polypeptide according to SEQ ID NO. 1 or 2 at a concentration ranging from 3 to 20 μg/ml. One example of islet cell culture medium is described as follows (and shown in Table 2 and Example 1): CMRL supplemented with L-glutamine (2 mmol), Ciprofloxacin (2 mg/L), Amphotericin B (0.1 mg/L) Penicillin (100,000 units/L) and Streptomycin (100,000 micrograms/L), and a polypeptide according to SEQ ID NO. 1 or 2 (at 10 μg/ml,) and Fetal calf serum (FCS) (10%) and human serum (10%). Control culture medium is described in Table 3.

Prior to transplantation, cells were cultured for approximately 50-60 days. At the time of transplant, cells were detached from the bottom of the plate/flask using trypsin (available from Gibco). Following filtration via a 40 μm sterile mesh, single cells were washed in a phosphate buffer solution (without Calcium and Magnesium), Hanks Balanced Salt Solution may also be used (both available from Sigma Aldrich) by spinning (from 180 g-300 g, for approximately 10 minutes) counted and resuspended in approximately 200 μl of sterile PBS (or HBSS) at a dose of 1.25×10⁶/100 μl and injected into anesthetized mice via the tail vein. Injection was carried out over the course of one minute.

Human insulin was detected in all mice on day 14 and day 30 (the concentration range was measured as 12.5 to 33 μmol/L) post first injection. Human C-peptide confirmed positive results when measured on day 30 (at a level up to 10 μmol).

As an alternative to intra venous injection, the cells can be resuspended in PBS at a more concentrated volume of 20-50 μl and inserted in the sub capsular space (occupying an area of approximately 1 cm²) of the kidney by using a PE-50 tubing connected to a syringe (method described by Bertera et al, Journal of Transplantation Volume 2012, Article ID 856386, 9 pages doi: 10.1155/2012/856386). Using this approach, larger doses of cells can be administered at once (for example, from about 5-10×10⁶ cells), in contrast to intravenous injection of human cells, of which doses higher than 2.5×10⁶ cells may not be as well tolerated.

Considering that normal range of insulin in the human serum of non-diabetic individuals is approximately 35.9 to 143.5 μmol/l, and considering the limitations derived from the difference in clearance of human insulin in mice compared to humans, it is reasonable to expect that a dose of 3.0 to 25×10⁶ cells/kg of body weight in human recipients would supply insulin in quantities that have the potential to affect glucose regulation. Administration may be once, or multiple administrations, repeated every 3 to 6 months, or on an annual basis, as needed. The dose will depend on many factors, such as severity of illness, gender, weight, and age.

### Example 5

Method of increasing insulin secretion in vivo via transplantation into a host animal of cellular compositions comprising insulin and glucagon secreting cells.

It has been determined that cellular compositions comprising insulin and glucagon secreting cells propagated from murine islets, using the islet cell culture medium described herein, can be safely injected and/or transplanted via the kidney capsule into animals, such as mice.

In one example, human insulin secretion following implantation of insulin and glucagon secreting cells under the kidney capsule in mice was detected for a duration of at least 100 days (See FIG. 4).

The insulin and glucagon secreting cells propagated were collected, then suspended in a solution, such as one comprising Hanks Balanced Salt Solution (HBSS) or Phosphate Buffered Saline (PBS) (available from Sigma Aldrich) to form a cellular composition. In this example, the cellular composition comprising insulin and glucagon secreting cells suspended in Hanks Balanced Salt Solution was transplanted under the kidney capsule of streptozotocin-diabetic (STZ) nude mice (5-6 weeks old; Jackson Laboratory, Bar Harbor, ME) using known methods, such as an approach described in Bertera et al 2012.

Briefly, prior to transplantation, mice were injected with streptozotocin (240 mg/kg IP) and hyperglycemia (non-fasting blood glucose levels >350 mg/dl on 2 consecutive readings) was confirmed.

On the day of transplantation, a cellular composition is made by detaching the insulin and glucagon secreting cells from culture, such as with trypsin; centrifuging (at 300 g) and counting the detached cells. Approximately 4×10⁶ cells; were suspended in a solution comprising HBSS; and loaded into a tubing or catheter, such as PE50 tubing. The cellular composition is then transplanted into an STZ mouse by placing the catheter or tubing containing the cellular composition under the kidney capsule of a fully anesthetized STZ mouse, via a small incision of the left flank, and following exposure of the kidney.

On days 14, 56 and 100 post-transplantation, blood was obtained from the tail vein of the STZ mice receiving the cellular compositions, and plasma was separated and stored. Insulin levels were measured using an ELISA kit specific for human insulin (ALPCO Diagnostics, Salem, NH, USA). See FIG. 4, showing insulin levels in the streptozotocin-diabetic mice treated, via transplantation, with a composition comprising insulin and glucagon secreting cells.

### Example 6

Method of treating hyperglycemia and diabetes via transplantation into a host animal of cellular compositions comprising insulin and glucagon secreting cells.

Cellular compositions comprising insulin and glucagon secreting cells generated by treating non-type 1 diabetic pancreatic tissue (human and murine in origin) with an islet cell culture medium comprising a base medium and a polypeptide according to SQ ID NO: 1 or 2 have been shown to secrete insulin following glucose stimulation at sufficient levels to lower blood glucose. These cellular compositions comprising approximately 4×10⁶ cells, when injected intravenously, not only secrete insulin and glucagon but home and engraft in the pancreas. Cellular compositions comprising insulin and glucagon secreting cells may be delivered via cell transplantation for the treatment of pancreatic disorders, including diabetes and hyperglycemia.

The expression of the stem cell marker CD133 correlates with the capacity of cells to engraft long-term, and these cells have the innate capability to migrate and home to injury sites. It was determined that cellular compositions as disclosed herein, when injected into an STZ-diabetic mouse, migrate to the pancreas and are shown to normalize hyperglycemia by a corresponding lowering of blood glucose levels following treatment. For example, six (6) four STZ-treated mice were injected with a cellular composition comprising (approximately) 20×10⁶ cells isolated from murine pancreatic tissue and treated with islet cell culture medium comprising a polypeptide according to SEQ ID NO. 1 or 2. Results showed that by day 10 all mice had lower blood glucose levels; by day 22 post injection two of the four animals exhibited a fasting blood glucose lower than 200 mg/dl. All recipient animals had a decrease in blood glucose levels, two of the animals-maintained blood glucose levels close to 250 mg/dl, with one animal having levels as low as 180 mg/dl on day 85 of the experiment. See FIG. 5.

As cells from normal pancreatic tissue display very similar characteristics to the cells generated from T1D pancreas samples, ie: triple positive for CD133, glucagon and insulin, it is reasonable to expect that the cellular compositions comprising insulin and glucagon secreting cells generated from a T1D pancreatic tissue sample treated with islet cell culture medium comprising a polypeptide according to SEQ NO: 1 or 2 will provide a measurable reduction in blood glucose levels in vivo. Moreover, implantations of cellular compositions comprising insulin and glucagon secreting cells have been shown to be non-oncogenic, making them a desirable option for treatment of pancreatic disorders by transplantation (by one of injection, infusion, engraftment).

### Example 7

Method of cultivation, viability testing and cryopreservation of insulin and glucagon secreting islet cells.

In one embodiment, an islet cell culture medium comprises CMRL-1066 (Mediatech, #99-663-CV; Transplant Medium (CMRL 1066) without phenol red) supplemented with: 10% Heat Inactivated Fetal Calf Serum (Gibco, #16140071), 10% Human Serum (Gemini, #100512), L-Glutamine (2 mM, Gibco, #25030081), Ciprofloxacin (2 ml/L, Bioworld, #403100313), Amphotericin-B (0.1 mg/L, Gibco, #15290026), Penicillin-Streptomycin (100,000 U/L-100,000 μg/L, Gibco, #10378016), and a peptide according to SEQ ID 1 or 2 (range of 3-20 g/ml, such as, 3 μg/ml, 5 μg/ml, or 10.0 μg/ml). In another embodiment, a cell culture medium for cryopreservation comprises a cryoprotectant medium (Gibco #12648010). In another embodiment, viability testing was carried out using methods comprising utilizing fluoroscein diacetate (FDA) (Sigma Aldrich #F7378) and propidium Iodide (PI) (Sigma Aldrich #P4170)

In one embodiment, human pancreatic tissue was collected via needle biopsy from human donors affected by Type 1 diabetes or severe pancreatitis and cultured in vitro in islet cell culture medium comprising CMRL 1066 supplemented with 10% fetal calf serum, 10% human serum, 2 mmol/L-glutamine, antibiotics, and a peptide according to SEQ ID NO 1 or 2 at a concentration range of 3 μg/mL to 20 μg/ml, such as 10.0 μg/mL, on plates (or flasks) coated with an attachment factor mixture (AFM) comprising Collagen Type I and Endothelial Cell Attachment Factor (ECAF). Various ratios of ECAF and collagen were used, including a 50/50 ratio of collagen to ECAF. A thin layer of AFM (between 3-10 ml) was applied and after setting for 30 minutes the excess AFM was removed; the plates were dried for 45 minutes in a hood. Plates are then washed with Phosphate Buffer Solution (PBS) to remove any potential contaminants. The collected pancreatic tissue was incubated on AFM-treated flasks/plates in islet cell culture medium for 10 to 20 days until cells began to mobilize and proliferate. Cultured cells are split when they reached confluency of approximately 70-80% in culture using standard aseptic techniques in a vertical laminar flow hood. The splitting technique involved removal of supernatant from the culture plates (supernatant was preserved). Plates are then washed with 2-5 ml PBS (wash was preserved). The cultured cells were detached using approximately 3-5 ml of trypsin (25% solution) by incubating the cells in the presence of trypsin at 37° C. for approximately 3-5 minutes until cells detached. The plate was then washed a second time with PBS. The trypsinized cells, as well as the preserved PBS washes and collected cell culture supernatant, were then centrifuged at 1000 rpm for 7 minutes at 4° C. The resulting supernatant was decanted and the pellet resuspended in 2 ml PBS, and centrifuged. The supernatant was then removed, and the pellet resuspended in islet cell culture medium comprising SEQ ID 1 or 2, and re-plated at a cell density of ~1000 cells/cm². Cultured cells that were not used for replating were resuspended at the maximum concentration of 1×10⁶/ml in a cryopreservation medium. Cultured cells were centrifuged (1000 rpm for 7 minutes) at 4° C. The supernatant was aspirated and replaced by fresh cryopreservation medium and the cells were transferred into a freezing container Mr. Frosty (Thermo-Fisher #5100-0036) overnight and then transferred and stored in vapor phase liquid nitrogen.

Cells were subject to a viability assay prior to cryopreservation. The minimal viability accepted for cryopreservation=95% (thus 95% of all cells analyzed were stained with FDA, viable fluorescence dye).

Two aliquots of approximately 200 cells were transferred in approximately 50 μl volume in a solution of 400 μl containing FDA and PI at the concentration of 0.46 μM for FDA and 14.34 μM for PI, in an Eppendorf tube. Cells were centrifuged (1000 rpm for 7 minutes) and approximately 95% of the Supernatant was aspirated and cells were transferred in the residual fluid in a microscope slide using a micropipette. Cells were analyzed under a fluorescence microscope (Olympus model CKX3) using a green/red filter set. Viable cells stain in green (FDA) and dead cells in red (PI). Percent of viable cells over total (expressed in percentage) was determined by two operators independently.

Although the foregoing invention has been described in some detail by way of illustration and example for purposes of clarity of understanding, it will be obvious that certain changes and modifications may be practiced within the scope of the appended claims. It will be clear to a person skilled in the art that features described in relation to any of the aspects and various embodiments described above can be applicable interchangeably between the different embodiments.

### ASPECTS

Aspect 1. A composition comprising an insulin and glucagon secreting cell population generated from non-insulin secreting pancreatic cells collected via needle biopsy from a Type 1 diabetic donor pancreas, a pancreatitis donor pancreas, or a combination thereof.

Aspect 2. The composition of Aspect 1, wherein at least about 50% of the insulin and glucagon secreting cell population expresses CD133, glucagon, and insulin.

Aspect 3. The composition of any one of Aspects 1-2, wherein about 50% to about 100% of insulin and glucagon secreting cell population expresses CD133, glucagon, and insulin, including all values in between, such as, for example, about 55%, about 60%, about 65%, about 70%, about 75%, about 80%, about 85%, about 90%, and about 95%; specify percent as a total of medium cell population).

Aspect 4. The composition of any one of Aspects 1-3, wherein the insulin and glucagon secreting cell population comprises from about 3×10⁶ cells to about 25×10⁶ cells per kg of body weight in a human recipient, including all values in between, such as, for example about 4×10⁶ cells, about 5×10⁶ cells, about 6×10⁶ cells, about 7×10⁶ cells, about 8×10⁶ cells, about 9×10⁶ cells, about 10×10⁶ cells, about 11×10⁶ cells, about 12×10⁶ cells, about 13×10⁶ cells, about 14×10⁶ cells, about 15×10⁶ cells, about 16×10⁶ cells, about 17×10⁶ cells, about 18×10⁶ cells, about 19×10⁶ cells, about 20×10⁶ cells, about 21×10⁶ cells, about 22×10⁶ cells, about 23×10⁶ cells, and about 24×10⁶ cells; where the mass (in kg) for typical human recipient depends on, for example, age height, and may range from about 4 kg to about 225 kg, including all values in between, such as about 10 kg, about 20 kg, about 30 kg, 4 about 0 kg, about 50 kg, about 60 kg, about 70 kg, about 80 kg, about 90 kg, about 100 kg, about 110 kg, about 120 kg, about 130 kg, about 140 kg, about 150 kg, about 160 kg, about 170 kg, about 180 kg, about 190 kg, about 200 kg, about 210 kg, and about 220 kg.

Aspect 5. The composition of any one of Aspects 1-4, wherein the non-insulin secreting pancreatic cells are collected via needle biopsy are obtained from a Type 1 diabetic donor pancreas.

Aspect 6. The composition of any one of Aspects 1-5, wherein the non-insulin secreting pancreatic cells collected via needle biopsy are obtained from a pancreatitis donor pancreas.

Aspect 7. The composition of any one of Aspects 1-6, wherein the non-insulin secreting pancreatic cells are isogenic, allogenic, or a combination thereof.

Aspect 8. A method of treating a pancreatic disorder, comprising administering to a subject in need thereof a therapeutically effective amount of the composition of any one of Aspects 1-7, wherein the pancreatic disorder comprises Type 1 diabetes, pancreatitis, or a combination thereof.

Aspect 9. The method of Aspect 8, wherein said administering comprises delivering to the subject the therapeutically effective amount of the composition of any one of Aspects 1-7 via one or more of an injection, infusion, omental or peritoneal pouch, surgical implantation, or via packaging the composition as part of a device to a target site in the subject.

Aspect 10. A method for preparing a composition comprising an insulin and glucagon secreting cell population, which comprises: treating in vitro a population of non-insulin secreting Type 1 diabetic pancreatic cells with an islet cell culture medium comprising a base medium and an effective amount of a polypeptide comprising SEQ ID NO. 1, an active fragment of SEQ ID No. 1, SEQ ID NO. 2, an active fragment of SEQ ID No. 2, or a combination thereof.

Aspect 11. The method of Aspect 10, wherein the treating further comprises differentiating the population of non-insulin secreting Type 1 diabetic pancreatic cells and propagating the insulin and glucagon secreting cell population.

Aspect 12. The method of Aspect 10, wherein the treating further comprises differentiating the population of non-insulin secreting Type 1 diabetic pancreatic cells and propagating the insulin and glucagon secreting cell population, wherein the insulin and glucagon secreting cell population at least about 50% of insulin and glucagon secreting cell population expresses CD133, glucagon, and insulin.

Aspect 13. The method of Aspect 10, wherein the treating further comprises differentiating the population of non-insulin secreting Type 1 diabetic pancreatic cells and propagating the insulin and glucagon secreting cell population, wherein the insulin and glucagon secreting cell population about 50% to about 100% of insulin and glucagon secreting cell population expresses CD133, glucagon, and insulin.

Aspect 14. The method of Aspect 10, wherein the treating further comprises differentiating the population of non-insulin secreting Type 1 diabetic pancreatic cells and propagating the insulin and glucagon secreting cell population, wherein the insulin and glucagon secreting cell population comprises from about 3×10⁶ cells to about 25×10⁶ cells per kg of body weight in a human recipient.

Aspect 15. The method of any one of Aspects 10-14 further comprising extracting the population of non-insulin secreting Type 1 diabetic pancreatic cells from a donor.

Aspect 16. The method of any one of Aspects 10-14 further comprising extracting the population of non-insulin secreting Type 1 diabetic pancreatic cells from a donor via needle biopsy.

Aspect 17. The method of any one of Aspects 10-16 further comprising extracting the population of non-insulin secreting Type 1 diabetic pancreatic cells from an isogenic donor, and allogenic donor, or a combination thereof.

Aspect 18. The method of any one of Aspects 10-17, wherein the islet cell culture medium comprises the polypeptide in amount that ranges from about 3 μg/mL to about 20 μg/mL and all values in between, such as, for example, about 4 μg/mL, about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, and about 19 μg/mL.

Aspect 19. The method of any one of Aspects 10-19, wherein the treating further comprises differentiating the population of non-insulin secreting Type 1 diabetic pancreatic cells and propagating the insulin and glucagon secreting cell population to obtain a therapeutically amount of the insulin and glucagon secreting cell population and the method further comprises administering to a subject in need thereof the therapeutically effective amount of the insulin and glucagon secreting cell population.

Aspect 20. A composition (e.g., any one of Aspects 1-7) comprising an insulin and glucagon secreting cell population generated from non-insulin secreting pancreatic cells collected via needle biopsy from a Type 1 diabetic donor pancreas for use in the treatment of Type 1 diabetes, pancreatitis, or a combination thereof.

Although the foregoing information highlights aspects disclosed herein by way of illustration and example for purposes of clarity of understanding, it will be obvious that certain changes and modifications may be practiced within the scope of the subject matter claimed herein. It will be clear to a person skilled in the art that features described in relation to any of the aspects and various embodiments described above can be applicable interchangeably between the different embodiments.

The aspects and embodiments described above are examples to illustrate various features of the subject matter claimed herein. All publications and patent applications disclosed herein are indicative of the level of those skilled in the art to which this disclosure and the subject matter of the claims pertains.

Throughout the description and claims of this specification, the words “comprise” and “contain” and variations of them mean “including but not limited to”, and they are not intended to (and do not) exclude other moieties, additives, components, or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, characteristics, compounds, chemical moieties, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The subject matter claimed herein is not restricted to the details of any foregoing embodiments. The subject matter claimed herein extends to any novel one, or any novel combination, of the features disclosed in this specification (including any accompanying claims, abstract and drawings), or to any novel one, or any novel combination, of the steps of any method or process so disclosed.

All publications and patent applications are herein incorporated by reference to the same extent as if each individual publication or patent application was specifically and individually to be incorporated by reference. Specific patent applications incorporated by reference include, for example, U.S. patent application Ser. No. 15/811,060, filed on Nov. 13, 2017 (and published as US 2018/0133280 A1); and International Patent Application No. PCT/US2019/038305 filed on Jun. 20, 2019 (and published as WO 2020/005721 A1). To the extent that terms and/or expressions incorporated herein conflict with the terms and/or expression disclosed herein, the information disclosed herein controls.

1. A composition comprising an insulin and glucagon secreting cell population generated from non-insulin secreting pancreatic cells collected from a Type 1 diabetic donor pancreas, a pancreatitis donor pancreas, or a combination thereof.

2. The composition of claim 1, wherein at least about 50% of the insulin and glucagon secreting cell population expresses CD133, glucagon, and insulin.

3. The composition of claim 1, wherein about 50% to about 100% of insulin and glucagon secreting cell population expresses CD133, glucagon, and insulin. (spec: including all values in between, such as, for example about 55%, 60%, 65%, 70%, 75%, 80%, 85%, 90%, and 95%; specify percent as a total of medium cell population).

4. The composition of claim 1, wherein the insulin and glucagon secreting cell population comprises from about 1.2×10⁶ cells to about 25×10⁶ cells per kg of body weight in a human recipient (spec: including all values in between, such as, for example about 1.5, 2, 3, 4, 5, 6, 7, 8, 9, 10, 11, 12, 13, 14, 15, 16, 17, 18, 19, 20, 21, 22, 23, and 24; specify mass (in kg) for typical human recipient (e.g., pediatric (e.g., 4-60 kg) and adult (60-225 kg)).

5. The composition of claim 1, wherein the non-insulin secreting pancreatic cells are collected from a Type 1 diabetic donor pancreas.

6. The composition of claim 1, wherein the non-insulin secreting pancreatic cells collected from a pancreatitis donor pancreas.

7. The composition of claim 1, wherein the non-insulin secreting pancreatic cells are isogenic, allogenic, or a combination thereof.

8. A method of treating a pancreatic disorder, comprising administering to a subject in need thereof a therapeutically effective amount of the composition of claim 1, wherein the pancreatic disorder comprises Type 1 diabetes, pancreatitis, or a combination thereof.

9. The method of claim 8, wherein said administering comprises delivering to the subject the therapeutically effective amount of the composition of claim 1 via one or more of an injection, infusion, omental or peritoneal pouch, surgical implantation, or via packaging the composition as part of a device to a target site in the subject.

10. A method for preparing a composition comprising an insulin and glucagon secreting cell population, which comprises:
treating in vitro a population of non-insulin secreting Type 1 diabetic pancreatic cells with an islet cell culture medium comprising a base medium and an effective amount of a polypeptide comprising an amino acid sequence having at least 80% sequence identity to the amino acid sequence set forth in SEQ ID NO. 1 or 2, a polypeptide comprising SEQ ID NO. 1, an active fragment of SEQ ID NO. 1, SEQ ID NO. 2, an active fragment of SEQ ID NO. 2, or a combination thereof.

11. The method of claim 10, wherein the treating further comprises differentiating the population of non-insulin secreting Type 1 diabetic pancreatic cells and propagating the insulin and glucagon secreting cell population.

12. The method of claim 10, wherein the treating further comprises differentiating the population of non-insulin secreting Type 1 diabetic pancreatic cells and propagating the insulin and glucagon secreting cell population, wherein the insulin and glucagon secreting cell population at least about 50% of insulin and glucagon secreting cell population expresses CD133, glucagon, and insulin.

13. The method of claim 10, wherein the treating further comprises differentiating the population of non-insulin secreting Type 1 diabetic pancreatic cells and propagating the insulin and glucagon secreting cell population, wherein the insulin and glucagon secreting cell population about 50% to about 100% of insulin and glucagon secreting cell population expresses CD133, glucagon, and insulin.

14. The method of claim 10, wherein the treating further comprises differentiating the population of non-insulin secreting Type 1 diabetic pancreatic cells and propagating the insulin and glucagon secreting cell population, wherein the insulin and glucagon secreting cell population comprises from about 3×10⁶ cells to about 25×10⁶ cells per kg of body weight in a human recipient.

15. The method of claim 10 further comprising extracting the population of non-insulin secreting Type 1 diabetic pancreatic cells from a donor.

16. The method of claim 10 further comprising extracting the population of non-insulin secreting Type 1 diabetic pancreatic cells from a donor via needle biopsy.

17. The method of claim 10 further comprising extracting the population of non-insulin secreting Type 1 diabetic pancreatic cells from an isogenic donor, and allogenic donor, or a combination thereof.

18. The method of claim 10, wherein the islet cell culture medium comprises the polypeptide in amount that ranges from about 3 μg/mL to about 20 μg/mL.

19. The method of claim 10, wherein the treating further comprises differentiating the population of non-insulin secreting Type 1 diabetic pancreatic cells and propagating the insulin and glucagon secreting cell population to obtain a therapeutically amount of the insulin and glucagon secreting cell population and the method further comprises administering to a subject in need thereof the therapeutically effective amount of the insulin and glucagon secreting cell population.

20. A composition comprising an insulin and glucagon secreting cell population generated from non-insulin secreting pancreatic cells collected from a Type 1 diabetic donor pancreas for use in the treatment of Type 1 diabetes, pancreatitis, or a combination thereof.
